(19) United States
(12) Patent Application Publication
(10) Pub. No.: US 2026/0279184 A1
(43) Pub. Date: Sep. 17, 2026

(54) METHOD AND SYSTEMS OF MOBILE DATA PROCESSING DEVICE BASED REMOTE MONITORING OF A FIREFIGHTER AIR REPLENISHMENT SYSTEM PROVIDING ACCESS TO BREATHABLE AIR

(71) Applicant: Rescue Air Systems, Inc., San Carlos, CA (US)

(72) Inventor: Anthony J. Turiello, Westlake, TX (US)

(73) Assignee: Rescue Air Systems, Inc., San Carlos, CA (US)

(21) Appl. No.: 19/681,899

(22) Filed: May 19, 2026

Related U.S. Application Data
(63) Continuation of application No. 18/139,357, filed on Apr. 26, 2023, now Pat. No. 12,658,019.
(60) Provisional application No. 63/356,996, filed on Jun. 29, 2022.
(60) Provisional application No. 63/359,882, filed on Jul. 11, 2022.

(51) Int. Cl. G08B 21/18; G01N 33/00; G08B 21/02

(52) U.S. Cl. CPC G08B 21/182; G01N 33/0031; G08B 21/02

(57) ABSTRACT

Disclosed are a method and systems of remote monitoring of a sensor integrated safety system of a structure through a mobile data processing device. In accordance therewith, the mobile data processing device executes a component of a computing platform for the sensor integration therethrough. The mobile data processing device executing the component of the computing platform monitors the safety system and/or one or more components of a number of components thereof remotely based on collecting a number of parameters of the number of components of the safety system and/or of access thereof through detection of the number of parameters via a set of sensors associated with the number of components of the safety system. The number of parameters includes one or more parameter(s) related to breathable air from a source within the safety system supplied thereacross via a fixed piping system implemented therein and/or access of the breathable air.

### CLAIM OF PRIORITY

This Application is a continuation of U.S. patent application Ser. No. 18/139,357, filed Apr. 26, 2023, which claims the benefit of and priority to U.S. Provisional Patent Application No. 63/356,996 titled CLOUD-BASED FIREFIGHTING AIR REPLENISHMENT MONITORING SYSTEM, SENSORS AND METHODS filed on Jun. 29, 2022 and U.S. Provisional Patent Application No. 63/359,882 titled REMOTE MONITORING AND CONTROL OF A FIREFIGHTER AIR REPLENISHMENT SYSTEM THROUGH SENSORS DISTRIBUTED WITHIN COMPONENTS OF THE FIREFIGHTER AIR REPLENISHMENT SYSTEM filed on Jul. 11, 2022. The contents of the aforementioned applications are incorporated herein by reference in entirety thereof.

### FIELD OF TECHNOLOGY

This disclosure relates generally to emergency systems and, more particularly, to a method and/or systems of mobile data processing device based remote monitoring of a safety system of a structure providing access to breathable air.

### BACKGROUND

A structure (e.g., a vertical building, a horizontal building, a tunnel, marine craft) may have a Firefighter Air Replenishment System (FARS) implemented therein. The FARS may have an emergency air fill station therein to enable firefighters and/or emergency personnel access breathable air therethrough. The FARS may have other components relevant to critical functioning thereof. However, tracking parameters (e.g., pressure of the breathable air supplied) of the FARS critical to the functioning and/or the maintenance thereof may be difficult due to a monolithic and/or a standalone implementation of the components of the FARS. In order to monitor the FARS, maintenance personnel may have to collect data from individual components thereof in a process that is tedious and/or inefficient.

### SUMMARY

Disclosed are a method and/or systems of mobile data processing device based remote monitoring of a safety system of a structure providing access to breathable air.

In one aspect, a method of a safety system of a structure having a fixed piping system implemented therein to supply breathable air from a source across the safety system is disclosed. The method includes integrating a computing platform executing on a data processing device with a set of sensors associated with a number of components of the safety system, and executing a component of the computing platform on a mobile data processing device communicatively coupled to the data processing device through a computer network. The method also includes, in accordance with the execution of the component of the computing platform on the mobile data processing device and the integration of the computing platform with the set of sensors, monitoring, through the mobile data processing device, the safety system and/or one or components of the number of components thereof remotely based on collecting a number of parameters of the number of components of the safety system and/or of access thereof through detection of the number of parameters via the set of sensors. The number of parameters includes one or more parameter(s) related to the breathable air and/or access of the breathable air.

In another aspect, a safety system of a structure having a fixed piping system implemented therein to supply breathable air from a source across the safety system is disclosed. The safety system includes a data processing device executing instructions associated with a computing platform thereon to integrate the computing platform with a set of sensors associated with a number of components of the safety system, and a mobile data processing device communicatively coupled to the data processing device through a computer network. The mobile data processing device executes instructions associated with a component of the computing platform thereon to, in accordance with the integration of the computing platform with the set of sensors, monitor the safety system and/or one or more component(s) of the number of components thereof remotely based on collecting a number of parameters of the number of components of the safety system and/or of access thereof through detection of the number of parameters via the set of sensors. The number of parameters includes one or more parameter(s) related to the breathable air and/or access of the breathable air.

In yet another aspect, a safety system of a structure having a fixed piping system implemented therein to supply breathable air from a source across the safety system is disclosed. The safety system includes a set of sensors associated with a number of components of the safety system, with the set of sensors integrated with a computing platform executing on a data processing device, and a mobile data processing device communicatively coupled to the data processing device through a computer network. The mobile data processing device executes a component of the computing platform thereon to, in accordance with the integration of the computing platform with the set of sensors, monitor the safety system and/or one or more component(s) of the number of components thereof remotely based on collecting a number of parameters of the number of components of the safety system and/or of access thereof through detection of the number of parameters via the set of sensors. The number of parameters includes one or more parameter(s) related to breathable air from a source within the safety system supplied thereacross via a fixed piping system implemented therein and/or access of the breathable air.

Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a schematic and an illustrative view of a safety system associated with a structure, according to one or more embodiments.

FIG. 2 is a schematic view of the safety system of FIG. 1 with elements thereof integrated therewithin in detail, according to one or more embodiments.

FIG. 3 is a schematic view of the air monitoring system of the safety system of FIGS. 1-2, according to one or more embodiments.

FIG. 4 is a schematic view of an emergency air fill station of the safety system of FIGS. 1-2, according to one or more embodiments.

FIG. 5 is a schematic view of an air storage system of the safety system of FIGS. 1-2 according to one or more embodiments.

FIG. 6 is a schematic view of a computing platform relevant to the safety system of FIGS. 1-2 implemented through a server, according to one or more embodiments.

FIG. 7 is a schematic view of a data processing device of FIGS. 2-6, according to one or more embodiments.

FIG. 8 is an example user interface view of a component of the computing platform of FIG. 6 executing on the data processing device of FIGS. 2-7.

FIG. 9 is another example user interface view of the component of the computing platform of FIG. 6 executing on the data processing device of FIGS. 2-7.

FIG. 10 is a dashboard view of the component of the computing platform of FIG. 6 executing on the data processing device of FIGS. 2-7.

FIG. 11 is a process flow diagram detailing the operations involved in mobile data processing device based remote monitoring of a safety system of a structure providing access to breathable air, according to one or more embodiments.

Other features of the present embodiments will be apparent from the accompanying drawings and from the detailed description that follows.

### DETAILED DESCRIPTION

Example embodiments, as described below, may be used to provide a method and/or systems of mobile data processing device based remote monitoring of a safety system of a structure providing access to breathable air. Although the present embodiments have been described with reference to specific example embodiments, it will be evident that various modifications and changes may be made to these embodiments without departing from the broader spirit and scope of the various embodiments.

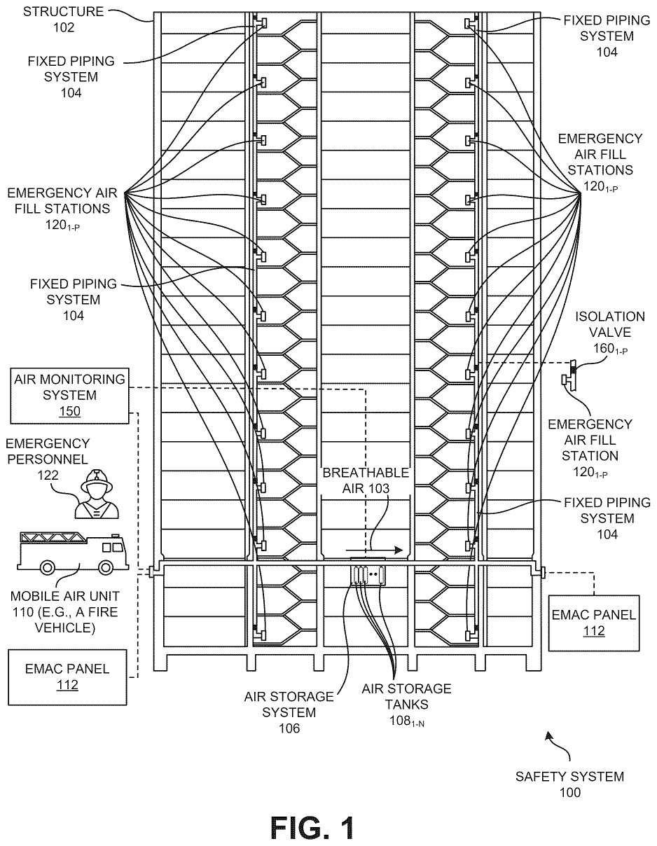

FIG. 1 shows a safety system 100 associated with a structure 102, according to one or more embodiments. In one or more embodiments, safety system 100 may be a Firefighter Air Replenishment System (FARS) to enable firefighters entering structure 102 in times of fire-related emergencies to gain access to breathable (e.g., human breathable) air (e.g., breathable air 103) in-house without the need of bringing in air bottles/cylinders to be transported up several flights of stairs of structure 102 or deep thereinto, or to refill depleted air bottles/cylinders that are brought into structure 102. In one or more embodiments, safety system 100 may supply breathable air provided from a supply of air tanks (to be discussed) stored in structure 102. When a fire department vehicle arrives at structure 102 during an emergency, breathable air supply typically may be provided through a source of air connected to said vehicle. In one or more embodiments, safety system 100 may enable firefighters to refill air bottles/cylinders thereof at emergency air fill stations (to be discussed) located throughout structure 102. Specifically, in some embodiments, firefighters may be able to fill air bottles/cylinders thereof at emergency air fill stations within structure 102 under full respiration in less than one to two minutes.

In one or more embodiments, structure 102 may encompass vertical building structures, horizontal building structures (e.g., shopping malls, hypermarts, extended shopping, storage and/or warehousing related structures), tunnels, marine craft (e.g., large marine vessels such as cruise ships, cargo ships, submarines and large naval craft, which may be “floating” versions of buildings and horizontal structures) and mines. Other structures are within the scope of the exemplary embodiments discussed herein. In one or more embodiments, safety system 100 may include a fixed piping system 104 permanently installed within structure 102 serving as a constant source of replenishment of breathable air 103. Fixed piping system 104 may be regarded as being analogous to a water piping system within structure 102 or another structure analogous thereto for the sake of imaginative convenience.

As shown in FIG. 1, fixed piping system 104 may distribute breathable air 103 across floors/levels of structure 102. For the aforementioned purpose, fixed piping system 104 may distribute breathable air 103 from an air storage system 106 (e.g., within structure 102) including a number of air storage tanks 108_{1-N} that serve as sources of pressurized/compressed air (e.g., breathable air 103). Additionally, in one or more embodiments, fixed piping system 104 may interconnect with a mobile air unit 110 (e.g., a fire vehicle) through an External Mobile Air Connection (EMAC) panel 112.

In one or more embodiments, EMAC panel 112 may be a boxed structure (e.g., exterior to structure 102) to enable the interconnection between mobile air unit 110 and safety system 100. For example, mobile air unit 110 may include an on-board air compressor to store and replenish pressurized/compressed air (e.g., breathable air analogous to breathable air 103) in air bottles/cylinders (e.g., utilizable with Self-Contained Breathing Apparatuses (SCBAs) carried by firefighters). Mobile air unit 110 may also include other pieces of air supply/distribution equipment (e.g., piping and/or air cylinders/bottles) that may be able to leverage the sources of breathable air 103 within safety system 100 through EMAC panel 112. Firefighters, for example, may be able to fill breathable air (e.g., breathable air 103, breathable air analogous to breathable air 103) into air bottles/cylinders (e.g., spare bottles, bottles requiring replenishment of breathable air) carried on mobile air unit 110 through safety system 100.

In FIG. 1, EMAC panel 112 is shown at two locations merely for the sake of illustrative convenience. In one or more embodiments, an air monitoring system 150 may be installed as part of safety system 100 to automatically track and monitor a parameter (e.g., pressure) and/or a quality (e.g., indicated by moisture levels, carbon monoxide levels) of breathable air 103 within safety system 100. FIG. 1 shows air monitoring system 150 as communicatively coupled to air storage system 106 and EMAC panel 112 merely for the sake of example. It should be noted that EMAC panel 112 may be at a remote location associated with (e.g., internal to, external to) structure 102. In one or more embodiments, for monitoring the parameters and/or the quality of breathable air within safety system 100, air monitoring system 150 include appropriate sensors and circuitries therein. For example, a pressure sensor (to be discussed) within air monitoring system 150 may automatically sense and record a pressure of breathable air 103 of safety system 100. Said pressure sensor may communicate with an alarm system that is triggered when the sensed pressure is outside a safety range. Also, in one or more embodiments, air monitoring system 150 may automatically trigger a shutdown of breathable air distribution through safety system 100 in case of impurity/contaminant (e.g., carbon monoxide) detection therethrough yielding levels above a safety/predetermined threshold.

In one or more embodiments, fixed piping system 104 may include pipes (e.g., constituted out of stainless steel tubing) that distribute breathable air 103 to a number of emergency air fill stations 120_{1-P} within structure 102. In one example implementation, each emergency air fill station 120_{1-P} may be located at a specific level of structure 102. If structure 102 is regarded as a vertical building structure, an emergency air fill station 120_{1-P} may be located at each of a basement level, a first floor level, a second floor level and so on. For example, emergency air fill station 120_{1-P} may be located at the end of the flight of stairs that emergency fighting personnel (e.g., firefighting personnel) need to climb to reach a specific floor level within the vertical building structure.

In one or more embodiments, an emergency air fill station 120_{1-P} may be a static location within a level of structure 102 that provides emergency personnel 122 (e.g., firefighters, emergency responders) with the ability to rapidly fill air bottles/cylinders (e.g., SCBA cylinders) with breathable air 103. In one or more embodiments, emergency air fill station 120_{1-P} may be an emergency air fill panel or a rupture containment air fill station. In one or more embodiments, proximate each emergency air fill station 120_{1-P}, safety system 100 may include an isolation valve 160_{1-P} to isolate a corresponding emergency air fill station 120_{1-P} from a rest of safety system 100. For example, said isolation may be achieved through the manual turning of isolation valve 160_{1-P} proximate the corresponding emergency air fill station 120_{1-P} or remotely (e.g., based on automatic turning) from air monitoring system 150. In one example implementation, air monitoring system 150 may maintain breathable air supply to a subset of emergency air fill stations 120_{1-P} via fixed piping system 104 through control of a corresponding subset of isolation valves 160_{1-P} and may isolate the other emergency air fill stations 120_{1-P} from the breathable air supply. It should be noted that configurations and components of safety system 100 may vary from the example safety system 100 of FIG. 1.

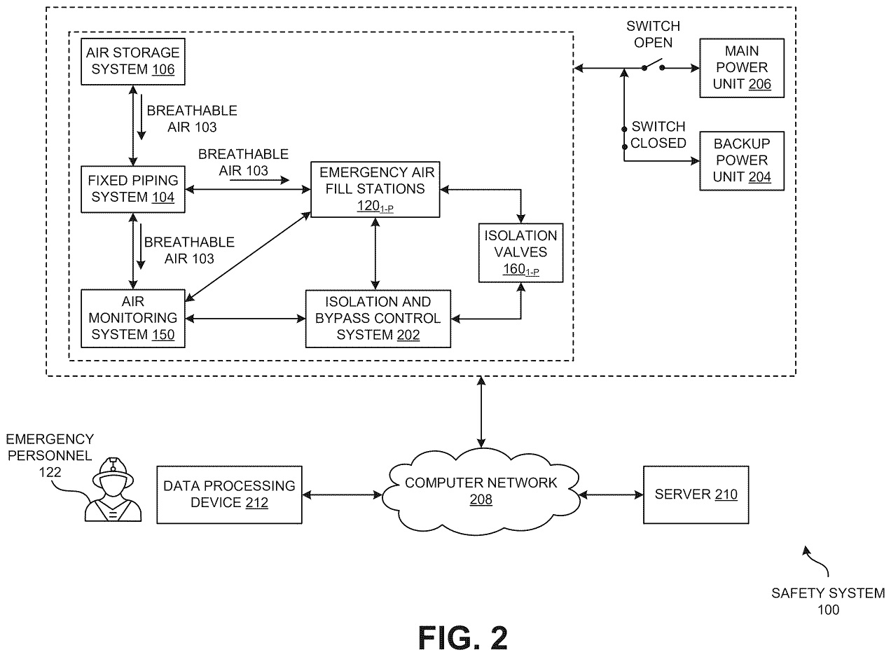

FIG. 2 shows safety system 100 with elements thereof integrated therewithin in detail, according to one or more embodiments. In one or more embodiments, safety system 100 may include air monitoring system 150 discussed above communicatively coupled to fixed piping system 104, to which emergency air fill stations 120_{1-P} are also coupled. In one or more embodiments, as seen above, the source of breathable air 103 may be air storage system 106. In one or more embodiments, safety system 100 may also include an isolation and bypass control system 202 that is constituted by a set of electrical, mechanical and/or electronic components working together to automatically include and/or bypass one or more emergency air fill station(s) 120_{1-P} based on detection of anomalous air parameters, as will be discussed below. For the aforementioned purpose, in one or more embodiments, isolation valve(s) 160_{1-P} associated with the aforementioned emergency air fill stations 120_{1-P} may be controlled (e.g., by opening or closing one or more of said isolation valves 160_{1-P}) by isolation and bypass control system 202.

Further, in one or more embodiments, safety system 100 may include a backup power unit 204 (e.g., an electrical power system with electronic integration) to ensure uninterrupted power to components of safety system 100 during emergencies (e.g., a power cut, a mains power issue, a fire accident effected power issue). For the aforementioned purpose, in one or more embodiments, backup power unit 204 may be switched on in the case of a power related emergency with respect to a main power unit 206 (e.g., Alternating Current (AC) mains power, Direct Current (DC) power) associated with safety system 100.

In one or more embodiments, one or more or all of the abovementioned components of safety system 100 may be integrated with sensor(s) to detect parameters of use therewithin. In one or more embodiments, one or more of the aforementioned components may be communicatively coupled through a computer network 208 (e.g., a Local Area Network (LAN), a Wide Area Network (WAN), the Internet, a cloud computing network, a short-range communication network based on Bluetooth®, WiFi® and the like) to a remote server 210 (e.g., a network of servers, a single server, a distributed network of servers, a command room server associated with safety system 100 and so on). As will be discussed below, in one or more embodiments, server 210 may obtain said parameters of use and other data from safety system 100 and perform analysis (e.g., predictive, non-predictive) thereof.

In addition, in one or more embodiments, safety system 100 may include a data processing device 212 (e.g., a mobile device such as a mobile phone, a tablet and an iPad®, a laptop, a desktop) also communicatively coupled to one or more components or each component of safety system 100 and server 210 through computer network 208. Thus, in one or more embodiments, one or more components or each component of safety system 100 may have interfaces (not explicitly shown) for wireless communication through computer network 208. Also, as will be discussed below, in one or more embodiments, wherever possible, elements (e.g., handheld Thermal Imaging Cameras (TICs), portable TICs, aerial TICs, video cameras, output audio devices, output light devices, one or more or all sensors discussed herein) may be Internet of Things (IoT) devices capable of collecting and feeding data to server 210 through computer network 208. In one or more embodiments, IoT devices (or IoT enabled devices) may be devices and/or components with programmable hardware that can transmit data over computer networks (e.g., computer network 208 such as the Internet and/or other networks); said IoT devices may include or be associated with edge devices (not shown) to control data flow at the boundaries to computer network 208.

It should be noted that both server 210 and data processing device 212 may both be remote from structure 102. In some embodiments, server 210 or data processing device 212 alone may be remote from structure 102. In some other embodiments, both server 210 and data processing device 212 may be within structure 102.

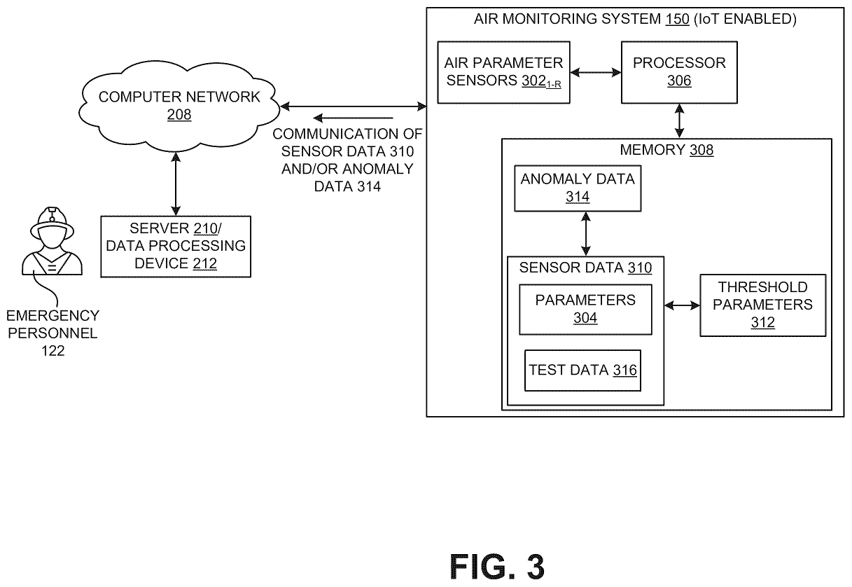

FIG. 3 shows air monitoring system 150, according to one or more embodiments. In one or more embodiments, air monitoring system 150 may include one or more air parameter sensors 302_{1-R} configured to sense parameters 304 associated with breathable air 103 such as pressure, temperature, oxygen content, carbon monoxide content, hydrocarbon content and moisture content; other parameters (e.g., air quality parameter(s), non-air quality parameter(s)) are within the scope of the exemplary embodiments discussed herein. In one or more embodiments, air monitoring system 150 may include a processor 306 (e.g., a microcontroller, a processor core, a single processor) communicatively coupled to a memory 308 (e.g., a volatile and/or a non-volatile memory); FIG. 3 shows air parameter sensors 302_{1-R} interfaced with processor 306. In one or more embodiments, data sensed by the aforementioned air parameter sensors 302_{1-R} may be part of sensor data 310 stored in memory 308; parameters 304 sensed may be part of sensor data 310.

In one or more embodiments, threshold values/ranges (e.g., threshold parameters 312) for parameters 304 sensed may also be stored in memory 308. In one or more embodiments, detecting through processor 306 in conjunction with one or more air parameter sensors 302_{1-R} that one or more parameters 304 is outside (e.g., below, above, outside) threshold parameters 312 may cause communication of anomalies (e.g., detected anomaly data 314 stored in memory 308) to server 210 through computer network 208 in accordance with the IoT capabilities discussed above. In one or more embodiments, threshold parameters 312 may be selected, controlled and/or set based on standards thereof such as National Fire Protection Association (NFPA) standards, Occupational Safety and Health Administration (OSHA) standards and/or Compressed Gas Association (CGA) standards.

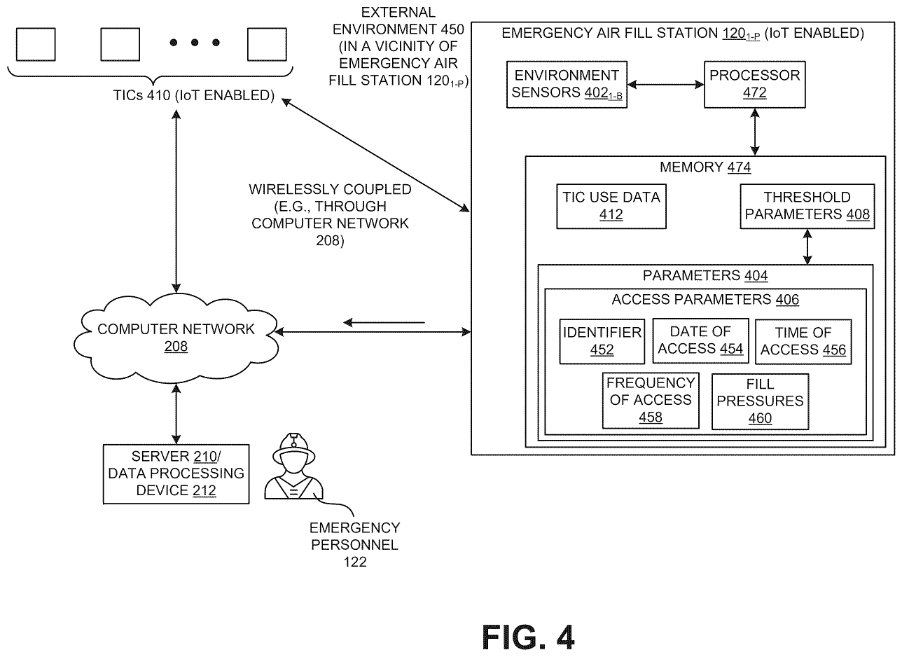

FIG. 4 shows an emergency air fill station 120_{1-P}, according to one or more embodiments. Again, in one or more embodiments, emergency air fill station 120_{1-P} may include one or more environment sensors 402_{1-B} integrated therewith configured to sense parameters 404 (e.g., temperature, ambient light) of an environment (e.g., external environment 450) in an immediate vicinity of emergency air fill station 120_{1-P}. In one or more embodiments, environment sensors 402_{1-B} may also sense access (e.g., access parameters 406 that are part of parameters 404 in FIG. 4) of emergency air fill station 120_{1-P} by emergency personnel 122 (e.g., maintenance personnel, firefighters, emergency responders). Example access parameters 406 may include but are not limited to identifier 452 of emergency personnel 122, date of access 454 mapped to identifier 452, time of access 456 mapped to identifier 452, a frequency of access 458 and fill pressures 460 (e.g., pressures to which breathable air 103 is filled in air bottles/cylinders discussed above) mapped to identifier 452, time of access 456 and/or date of access 454.

In one or more embodiments, again based on sensed parameters 404 being outside (e.g., more than, less than, outside a range) threshold values/ranges (e.g., threshold parameters 408) based on the IoT capabilities discussed herein, anomalies in parameters 404 may be detected and collected at emergency air fill station 120_{1-P} and transmitted to server 210 through computer network 208. In one or more embodiments, as shown in FIG. 4, emergency air fill station 120_{1-P} may include a processor 472 (e.g., a microcontroller, a processor core, a single processor) communicatively coupled to a memory 474 (e.g., a volatile and/or a non-volatile memory). In one or more embodiments, environment sensors 402_{1-B} may be interfaced with processor 472 and all of the abovementioned data/parameters may be stored in memory 474, as shown in FIG. 4.

FIG. 4 also shows TICs 410 as part of safety system 100 and in external environment 450 of emergency air fill station 120_{1-P}, according to one or more embodiments. In one or more embodiments, TICs 410 may be infrared cameras that sense infrared energy of objects to render images/video frames thereof corresponding to surface temperatures of said objects. In one or more embodiments, emergency personnel 122 may employ said TICs 410 to detect obstacles on the paths to/around emergency air fill stations 120_{1-P} under low visibility; this may enable emergency personnel 122 perform rescue operations efficiently. As discussed and implied above, TICs 410 may be integrated with IoT capabilities to transmit data to server 210 through computer network 208. Said data may be part of access parameters 406 or separate data (e.g., TIC use data 412) transmitted to server 210.

It should be noted that the sensing, detection and/or transmission of data to server 210 discussed above with regard to emergency air fill station 120_{1-P} may also be performed at a device external to emergency air fill station 120_{1-P}. In such implementations, the external device itself may obviously be a component of safety system 100 with IoT/wireless communication capabilities. All reasonable variations are within the scope of the exemplary embodiments discussed herein.

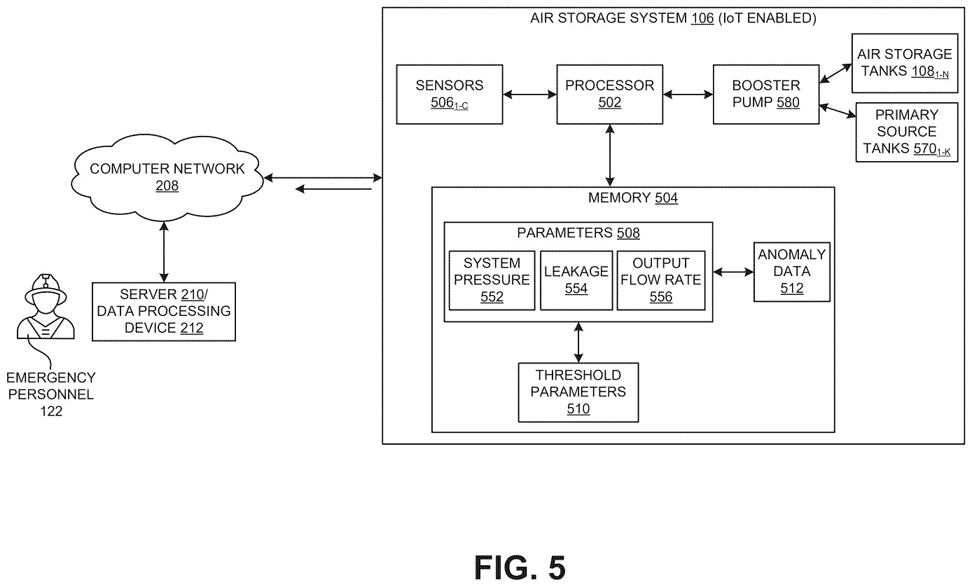

FIG. 5 shows air storage system 106, according to one or more embodiments. Again, as discussed above, in one or more embodiments, air storage system 106 may have IoT/wireless communication capabilities embedded therein or in a device external thereto that is communicatively coupled to air storage system 106. In one or more embodiments, air storage system 106 may include a processor 502 (e.g., a microcontroller, a processor core, a single processor) communicatively coupled to a memory 504 (e.g., a volatile and/or a non-volatile memory). Again, in one or more embodiments, air storage system 106 may include one or more sensors 506_{1-C} configured to sense parameters (e.g., parameters 508 stored in memory 504) associated with air storage system 106; sensors 506_{1-C} are shown interfaced with processor 502 Example parameters 508 sensed may include but are not limited to system pressure 552 (e.g., pressure at which breathable air 103 is output from air storage system 106), leakage 554 (e.g., leakage of breathable air 103 from air storage tanks 108_{1-N}) and output flow rate 556 (e.g., rate of flow of breathable air 103 out of air storage system 106). In one or more embodiments, parameters 508 may be transmitted to server 210 through computer network 208 for processing and/or analysis thereat.

Again, in one or more embodiments, anomalies based on parameters 508 being outside thresholds/ranges (e.g., threshold parameters 510 stored in memory 504) may be detected through sensors 506_{1-C} (e.g., flow rate sensors, pressure sensors). FIG. 5 shows anomaly data 512 relevant to the aforementioned detected anomalies also transmitted to server 210 through computer network 208, according to one or more embodiments.

It should be noted that FIGS. 3-5 merely relate to example components of safety system 100 with which sensors/IoT devices are integrated and that integration of sensors/IoT devices with any other component (e.g., backup power unit 204 to sense frequency and/or duration of use thereof, isolation and bypass control system 202 to sense a frequency of bypass/isolation of emergency air fill stations 120_{1-P}, turning on/off of isolation valves 160_{1-P} and so on) thereof conceivable is within the scope of the exemplary embodiments discussed herein. Referring back to FIG. 4, identifier 452 within access parameters 406 relevant to access of emergency air fill station 120_{1-P} may also encompass a key fob based identification, a Radio Frequency Identification (RFID) based access, a Non-Fungible Token (NFT) based access, keys and/or access through an application component (e.g., component 706 to be discussed below) executing on data processing device 212. All reasonable variations are within the scope of the exemplary embodiments discussed herein.

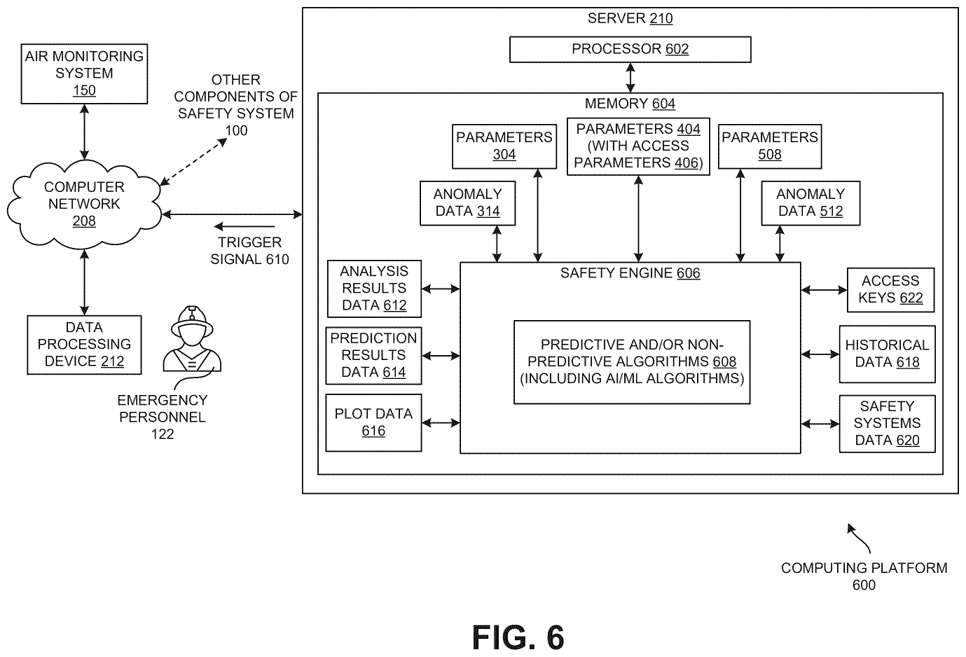

FIG. 6 shows a computing platform 600 relevant to the FARS of safety system 100 implemented through server 210, according to one or more embodiments. In one or more embodiments, server 210 may be a distributed (e.g., across a cloud) network of servers, a cluster of servers or a standalone server. As shown in FIG. 6, server 210 may include a processor 602 (e.g., a processor core, a network of processors, a single processor), communicatively coupled to a memory 604 (e.g., a volatile and/or a non-volatile memory). In one or more embodiments, memory 604 may include a safety engine 606 associated with the FARS stored therein and executable through processor 602. FIG. 6 shows memory 604 as including data (e.g., detected, sensed, anomalies) from one or more components of safety system 100; the limited amount of data shown must not be considered as limiting the scope of the exemplary embodiments discussed herein. In one or more embodiments, safety engine 606 may have one or more predictive and/or non-predictive algorithms (e.g., predictive and/or non-predictive algorithms 608) including Artificial Intelligence (AI)/Machine Learning (ML) based algorithms stored therein.

In one or more embodiments, execution of predictive and/or non-predictive algorithms 608 through processor 602 may involve taking the abovementioned data and profiling the FARS implemented as safety system 100. It should be noted that each of the aforementioned data (e.g., parameters 304, parameters 404, access parameters 406, parameters 508, anomaly data 314, anomaly data 512) may be real-time data from elements of safety system 100. In one or more embodiments, analysis of the data may result in beneficial decision making with regard to maintenance of safety system 100, safety of safety system 100 and/or efficiency thereof. For example, anomalies discussed above may be analyzed based on date, time and/or frequency thereof to predict that a specific duration of time in a winter season is associated with diminished characteristics of a component of safety system 100. All possible analyses are within the scope of the exemplary embodiments discussed herein.

In one or more embodiments, server 210 may also be utilized to remotely test and/or trigger operations of one or more components of safety system 100. FIG. 6 shows a trigger signal 610 communicated to air monitoring system 150 to get data thereof discussed above from processor 306, according to one or more embodiments. In some implementations, the components of safety system 100 may automatically transmit data thereof to server 210 and in some others, server 210 may transmit trigger signals (e.g., trigger signal 610) therefor. FIG. 6 also shows results of analysis/prediction through safety engine 606 as analysis results data 612, prediction results data 614 and plot data 616 (e.g., related to graphically plotting the results of analyses). Further, FIG. 6 shows data processing device 212 communicatively coupled to server 210 through computer network 208 as part of computing platform 600, according to one or more embodiments.

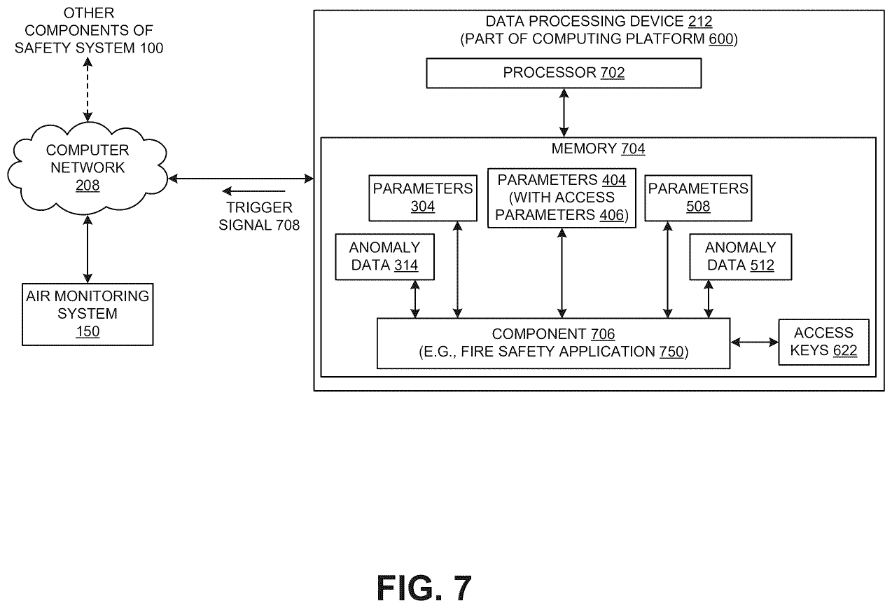

FIG. 7 shows data processing device 212 (e.g., a mobile phone, a tablet, a smart device, a laptop) in detail, according to one or more embodiments. In one or more embodiments, again, data processing device 212 may include a processor 702 (e.g., a single processor, a processor core) communicatively coupled to a memory 704 (e.g., a volatile and/or a non-volatile memory). In one or more embodiments, memory 704 may include a component 706 of safety engine 606 stored therein and enabled/provided through processor 602 of server 210. FIG. 7 shows component 706 as a fire safety application 750 merely for example purposes. Again, in one or more embodiments, access to the data of one or more components of safety system 100 may be available to data processing device 212 via component 706 (e.g., through computer network 208 via safety engine 606 of server 210). FIG. 7 also shows capabilities to control components of safety system 100 through data processing device 212 via trigger signals; FIG. 7 specifically shows a trigger signal 708 to initiate collection of data from air monitoring system 150 merely for example purposes. Again, in some implementations, data may be automatically communicated to data processing device 212 and in some others, data processing device 212 may trigger (e.g., through trigger signal 708) collection thereof.

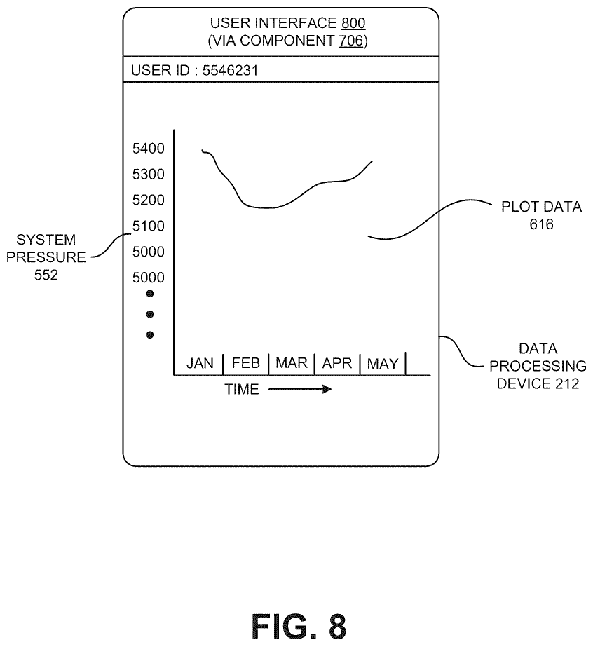

In one or more embodiments, access of emergency air fill station 120_{1-P} through component 706 may cause collection of identifier 452 discussed above as part of access parameters 406. FIG. 8 shows an example user interface 800 provided via component 706 (e.g., fire safety application 750) executing on data processing device 212. Here, user interface 800 shows plot data 616 discussed above that may be based on one or more examples of data discussed above and/or analysis results data 612; plot data 616 in FIG. 8 is an evolution of system pressure 552 over a scale of time. Obviously, emergency personnel 122 and/or other users associated with data processing device 212 may access user interface 800 after authentication thereof via computing platform 600 and/or navigating to user interface 800. All reasonable variations are within the scope of the exemplary embodiments discussed herein.

Thus, exemplary embodiments discussed herein provide for an integrated FARS computing platform (e.g., computing platform 600) that enables collection and/or analysis of real-time data from one or more components of safety system 100 and/or control (e.g., remotely) thereof. Further, the integrated FARS computing platform may provide for profiling of safety system 100 and/or emergency personnel 122 and/or remote management of requirements associated with safety system 100. For example, the profiling may involve utilizing (e.g., through safety engine 606) historical data (e.g., historical data 618 stored in memory 604 of server 210) from one or more components of safety system 100 and/or generic safety systems data (e.g., safety systems data 620 stored in memory 604 of server 210) from one or more safety systems other than safety system 100 to arrive at parts of analysis results data 612, prediction results data 614 and/or plot data 616. Again, as discussed above, in one or more embodiments, the integrated FARS computing platform may provide for quick decision making on the part of maintenance personnel, administrative personnel and/or emergency personnel (e.g., emergency personnel 122) associated with safety system 100; statistical analyses and/or data gathering and/or predictive and/or non-predictive analyses may also be enabled through the integrated FARS computing platform.

Also, in one or more embodiments, analogous analyses and/or prediction may also be performed at data processing device 212 based on enablement thereof through component 706. Further, it should be noted that detection of anomalies (e.g., anomaly data 314, anomaly data 512) may be performed through server 210 based on execution of safety engine 606 discussed above instead of or in addition to the detection thereof at the respective components. Last but not the least, as computing platform 600 may be enabled through the execution of safety engine 606, which, in turn, may enable component 706, both safety engine 606 and component 706 may be interpreted as computing platform 600 executing on server 210 and data processing device 212 respectively. In some scenarios, safety engine 606 and component 706 and server 210 and data processing device 212 corresponding thereto may be the same. All reasonable variations are within the scope of the exemplary embodiments discussed herein.

It should be noted that, in some embodiments, server 210 may be implemented in conjunction with a control room (not shown) within structure 102 or external thereto. In one or more embodiments, said control room may be associated with administrative personnel, maintenance personnel, security personnel and/or emergency responders, all of which may be examples of emergency personnel 122. In some implementations, server 210 (or, the control room) may authenticate emergency personnel 122 to enable access thereof to various components of safety system 100. For example, emergency personnel 122 may obtain a temporary identification pass from the control room; in another example, server 210 may assign an access key to emergency personnel 122 implementable/enabled through component 706/fire safety application 750. The IoT integration discussed herein may enable emergency personnel 122 gain access to one or more components (e.g., air monitoring system 150, emergency air fill station 120_{1-P}, air storage system 106) of safety system 100 through the assigned access key. FIGS. 6-7 show access keys 622 including the aforementioned generated access key stored in memory 604 and memory 704 of server 210 and data processing device 212 respectively.

In one example implementation, emergency personnel 122 may carry data processing device 212 thereof to a specific component (say, emergency air fill station 120_{1-P}) of safety system 100. The IoT capability discussed above with regard to emergency air fill station 120_{1-P} may enable emergency air fill station 120_{1-P} to authenticate emergency personnel 122 to provide access thereof based on reading access key 622 from fire safety application 750 executing on data processing device 212 wirelessly. Here, server 210 executing safety engine 606 may have configured emergency air fill station 120_{1-P} (e.g., processor 472) with the capability to read access key 622 from data processing device 212. Alternatively or additionally, server 210 may dynamically authenticate emergency personnel 122 based on proximity of data processing device 212 thereof to emergency air fill station 120_{1-P} automatically triggering transmission of a message to server 210 from component 706/emergency air fill station 120_{1-P}; server 210, in turn, may transmit a trigger signal (e.g., trigger signal 610) to emergency air fill station 120_{1-P} to provide emergency personnel 122 access thereto. In yet another implementation, emergency personnel 122 may (remotely, non-remotely) self-authenticate based on entering access key 622 thereof via fire safety application 750.

Referring back to air storage system 106 and FIG. 5, in addition to air storage tanks 108_{1-N}, in some implementations, air storage system 106 may include one or more primary source tanks 570_{1-K} in which breathable air 103 is maintained at an appropriate pressure (e.g., 5500 Pounds per Square Inch (PSP), 4500 PSI). In one or more embodiments, these primary source tanks 5701-K may be coupled to air storage tanks 108_{1-N} and may be utilized to provide breathable air 103 distributed via fixed piping system 104 and accessible via emergency air fill stations 120_{1-P}. In one or more embodiments, breathable air 103 from air storage tanks 108_{1-N} may flow to primary source tanks 570_{1-K} in a regulated manner such that a pressure of breathable air 103 within primary source tanks 570_{1-K} may be at an optimum level required to fill air bottles/cylinders of SCBAs. In one or more embodiments, a booster pump 580 may be coupled between air storage tanks 108_{1-N} and primary source tanks 570_{1-K} to enable transfer (e.g., enhanced/pressure-boosted transfer) of breathable air 103 from air storage tanks 108_{1-N} to primary source tanks 570_{1-K} as and when required (e.g., when breathable air 103 in primary source tanks 570_{1-K} is depleted, when pressure of breathable air 103 in primary source tanks 570_{1-K} drops below a threshold level thereof). In one or more embodiments, control of booster pump 580 and calibration thereof may be effected through sensors 506_{1-C} (e.g., tracking depletion/pressure of breathable air 103 in air storage tanks 108_{1-N}) and processor 502 and/or remotely through server 210/data processing device 212.

Referring back to air monitoring system 150 and FIG. 3, air parameter sensors 302_{1-R} may also be configured to collect test data 316 (e.g., real-time) from breathable air 103 based on trigger signals (e.g., trigger signal 610, trigger signal 708) from server 210/data processing device 212. In other words, emergency personnel 122 at server 210/data processing device 212 may also trigger the automatic collection of test data 316 (e.g., stored in memory 308 and transmittable to memory 604/704 of server 210/data processing device 212) from breathable air 103. For the aforementioned purpose, in one or more embodiments, breathable air 103 may pass through a specific collection chamber (not shown) within air monitoring system 150 and parameters 304 thereof collected as part of test data 316. In some implementations, air monitoring system 150 may be automatically configured through server 210/safety engine 606 to collect test data 316 as part of maintenance, analysis and/or regulatory requirements of safety system 100.

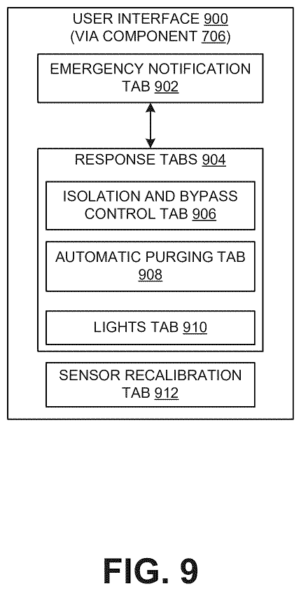

FIG. 9 shows another example user interface 900 provided via component 706 (e.g., fire safety application 750) executing on data processing device 212. Here, user interface 900 may, in conjunction with safety engine 606/component 706, provide an emergency notification (e.g., emergency situations such as a fire, polluted breathable air 103 and so on) via an emergency notification tab 902 thereof. Emergency personnel 122 may be provided the opportunity to respond to said emergency notification through response tabs 904 such as an isolation and bypass control tab 906 to control isolation and bypass control system 202 to exclude one or more emergency air fill stations 120_{1-P}, an automatic purging tab 908 to automatically purge breathable air 103 through safety system 100 and a lights tab 910 to automatically turn on lights in areas associated with emergency air fill station(s) 120_{1-P} to aid emergency personnel 122 in rescue operations thereof. Other response tabs 904 are within the scope of the exemplary embodiments discussed herein. Other options such as a sensor recalibration tab 912 to recalibrate one or more sensor(s) within one or more components of safety system 100 are also shown as part of user interface 900.

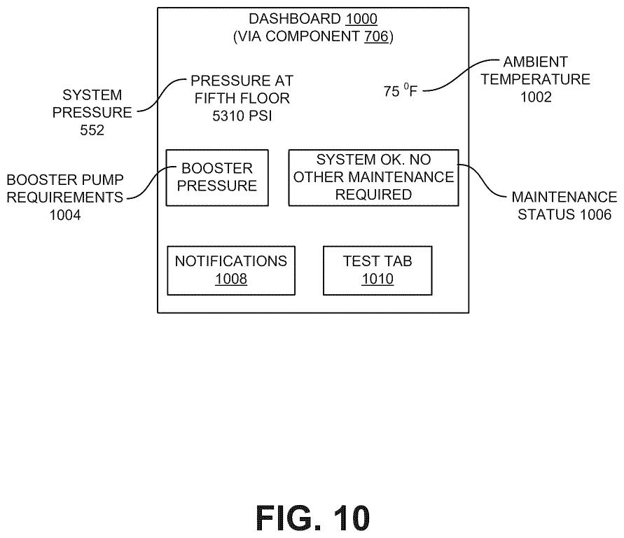

FIG. 10 shows an example dashboard 1000 (another user interface) shown via component 706 (e.g., fire safety application 750) of data processing device 212. Here, dashboard 1000 may show system pressure 552 of breathable air 103 at a current floor level (e.g., fifth floor) of structure 102, ambient temperature 1002 (e.g., sensed through environment sensors 402_{1-B}), booster pump requirements 1004 (e.g., requirement(s) to activate booster pump 580 to supply enhanced breathable air 103 to primary source tanks 570_{1-K}) and so on. Dashboard 1000 may also show maintenance status 1006 of safety system 100 and notifications 1008 relevant to proper maintenance thereof. Dashboard 100 may further show a test tab 1010 to trigger testing of breathable air 103 through safety system 100. Other example tabs and components of dashboard 1000 are within the scope of the exemplary embodiments discussed herein.

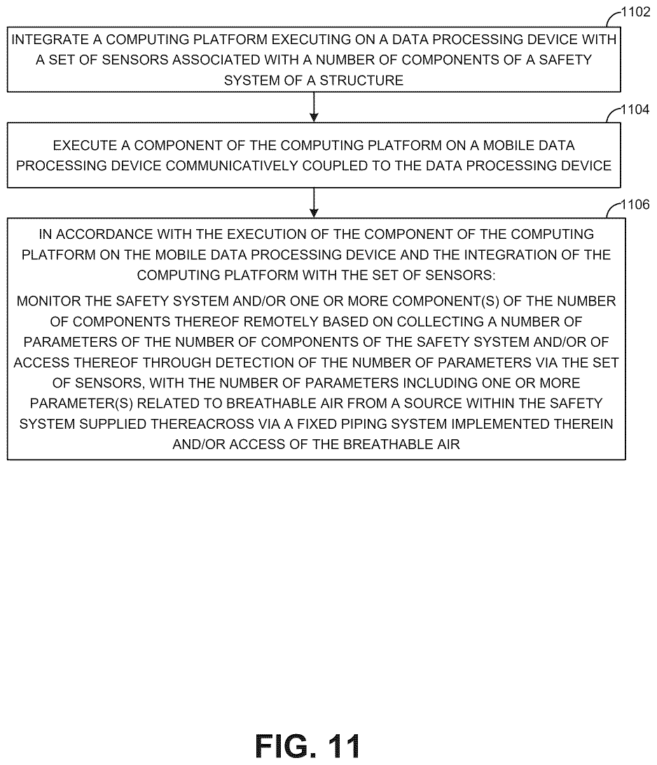

FIG. 11 shows a process flow diagram detailing the operations involved in mobile data processing device based remote monitoring of a safety system (e.g., safety system 100) of a structure (e.g., structure 102) providing access to breathable air (e.g., breathable air 103) from a source (e.g., air storage system 106) via a fixed piping system (e.g., fixed piping system 104) implemented therein, according to one or more embodiments. In one or more embodiments, operation 1102 may involve integrating a computing platform (e.g., computing platform 600, safety engine 606) executing on a data processing device (e.g., server 210) with a set of sensors (e.g., air parameter sensors 302_{1-R}, environment sensors 402_{1-B}, sensors 506_{1-C}) associated with a number of components (e.g., air monitoring system 150, emergency air fill station 120_{1-P}, air storage system 106) of the safety system. In one or more embodiments, operation 1104 may involve executing a component (e.g., component 706) of the computing platform on a mobile data processing device (e.g., data processing device 212) communicatively coupled to the data processing device through a computer network (e.g., computer network 208).

In one or more embodiments, operation 1106 may then involve, in accordance with the execution of the component of the computing platform on the mobile data processing device and the integration of the computing platform with the set of sensors, monitoring, through the mobile data processing device, the safety system and/or one or more components of the number of components thereof remotely based on collecting a number of parameters (e.g., parameters 304, parameters 404, parameters 508) of the number of components of the safety system and/or of access (e.g., access parameters 406) thereof through detection of the number of parameters via the set of sensors. In one or more embodiments, the number of parameters may include one or more parameter(s) (e.g., system pressure 552, leakage 554, output flow rate 556, fill pressures 460) related to the breathable air and/or access of the breathable air.

Although the present embodiments have been described with reference to specific example embodiments, it will be evident that various modifications and changes may be made to these embodiments without departing from the broader spirit and scope of the various embodiments.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1-20. (canceled)

21. A firefighter air replenishment system (FARS), comprising:
a safety system, comprising:
a source of air comprising tanks storing air at at least 4500 pounds per square inch (PSI); and
a fixed piping system coupled with the source of air;
a plurality of sensors coupled with the safety system; and
a data processing device coupled with the plurality of sensors, the data processing device comprising one or more processors to:
receive sensor data from the plurality of sensors;
authenticate access to the data processing device based at least on an identifier of a user; and
present an output based at least on the sensor data and the authentication of access to the data processing device.

22. The FARS of claim 21, comprising:
the one or more processors are to store, in association with the identifier of the user, an access record comprising at least one of a date of access, a time of access, a frequency of access, or a fill pressure associated with access to an emergency air fill station.

23. The FARS of claim 21, comprising:
the output comprises an access history for an emergency air fill station generated based at least on the identifier of the user and sensor data associated with the emergency air fill station.

24. The FARS of claim 21, comprising:
the plurality of sensors comprise one or more air parameter sensors to sense at least one of pressure, temperature, oxygen content, carbon monoxide content, hydrocarbon content, or moisture content of breathable air in the safety system.

25. The FARS of claim 21, comprising:
the output comprises an air-quality indication based on the sensor data comprising at least one of pressure, temperature, oxygen content, carbon monoxide content, hydrocarbon content, or moisture content.

26. The FARS of claim 21, comprising:
the one or more processors are to:
compare the sensor data to one or more predetermined thresholds; and
present, as the output, an anomaly indication based at least on the sensor data not satisfying the one or more predetermined thresholds.

27. The FARS of claim 21, comprising:
the one or more processors are to detect at least one emergency air fill station to be isolated from a breathable-air supply by an isolation and bypass control system that controls one or more isolation valves associated with the at least one emergency air fill station.

28. The FARS of claim 21, comprising:
the plurality of sensors comprise one or more air storage system sensors to sense at least one of system pressure, leakage, or output flow rate of breathable air from an air storage system of the safety system.

29. The FARS of claim 21, comprising:
the one or more processors are to present, as the output, a booster pump requirement based on the sensor data indicating a pressure of breathable air in one or more air storage tanks or one or more primary source tanks.

30. The FARS of claim 21, comprising:
the one or more processors are to generate a profile of the safety system based on historical data, and to present the output based on the generated profile.

31. A method, comprising:
receiving, by one or more processors of a data processing device coupled with a plurality of sensors coupled with a safety system, sensor data from the plurality of sensors, the safety system comprising a source of air and a fixed piping system coupled with the source of air;
authenticating, by the one or more processors, access to the data processing device based at least on an identifier of a user; and
presenting, by the one or more processors, an output based at least on the sensor data and the authentication of access to the data processing device.

32. The method of claim 31, comprising:
storing, by the one or more processors and in association with the identifier of the user, an access record comprising at least one of a date of access, a time of access, a frequency of access, or a fill pressure associated with access to an emergency air fill station.

33. The method of claim 31, comprising:
presenting, as the output, an access history for an emergency air fill station generated based at least on the identifier of the user and sensor data associated with the emergency air fill station.

34. The method of claim 31, comprising:
sensing, by one or more air parameter sensors of the plurality of sensors, at least one of pressure, temperature, oxygen content, carbon monoxide content, hydrocarbon content, or moisture content of breathable air in the safety system.

35. The method of claim 31, comprising:
presenting, as the output, an air-quality indication based on the sensor data comprising at least one of pressure, temperature, oxygen content, carbon monoxide content, hydrocarbon content, or moisture content.

36. The method of claim 31, comprising:
comparing, by the one or more processors, the sensor data to one or more predetermined thresholds; and
presenting, as the output, an anomaly indication based at least on the sensor data not satisfying the one or more predetermined thresholds.

37. The method of claim 31, comprising:
detecting, by the one or more processors, at least one emergency air fill station to be isolated from a breathable-air supply by an isolation and bypass control system that controls one or more isolation valves associated with the at least one emergency air fill station.

38. The method of claim 31, comprising:
sensing, by one or more air storage system sensors of the plurality of sensors, at least one of system pressure, leakage, or output flow rate of breathable air from an air storage system of the safety system.

39. The method of claim 31, comprising:
presenting, by the one or more processors and as the output, a booster pump requirement based on the sensor data indicating a pressure of breathable air in one or more air storage tanks or one or more primary source tanks.

40. The method of claim 31, comprising:
generating, by the one or more processors, a profile of the safety system based on historical data, and presenting the output based on the generated profile.
